(19) United States
(12) Patent Application Publication
(10) Pub. No.: US 2004/0156831 A1
(43) Pub. Date: Aug. 12, 2004

(54) Bacteriophage having modified holin and uses thereof

(76) Inventors: Janakiraman Ramachandran, Palo Alto, CA (US); Sriram Padmanabhan, Bangalore (IN); Bharathi Sriram, Bangalore (IN)

Correspondence Address: BOZICEVIC, FIELD & FRANCIS LLP, 200 MIDDLEFIELD RD, SUITE 200, MENLO PARK, CA, 94025, US

(21) Appl. No.: 10/714,591

(22) Filed: Nov. 14, 2003

Related U.S. Application Data
(60) Provisional application No. 60/426,687, filed on Nov. 14, 2002.

(51) Int. Cl. A61K 45/00

(52) U.S. Cl. 424/93.6

(57) ABSTRACT

The present invention features composition and methods for treating a bacterial infection using therapeutic bacteriophage having a modified holin gene. The modified holin inactivates the bacterial host prior to production of bacteriophage, so that the bacteriophage infection is non-productive, e.g., few or no bacteriophage are produced as a result of infection of the bacterial host. Thus, holin-modified bacteriophage invade the bacterial host, and cause inactivation of the bacterial host prior to production of a detectable or significant number of phage. Holin-modified phage inhibit the spread of bacterial infection without production of a significant or detectable number of phage progeny. By avoiding the release of phage progeny, the potential for generation of immune responses against the phage is reduced.

### CROSS-REFERENCE TO RELATED APPLICATIONS

This application claims the benefit of U.S. provisional application serial No. 60/426,687, filed Nov. 14, 2002, which application is incorporated herein by reference in its entirety.

### FIELD OF THE INVENTION

The invention relates to methods and compositions for treatment of bacterial infections, particularly therapeutic bacteriophages, particularly therapeutic bacteriophages having reduced immunogenicity.

### BACKGROUND OF THE INVENTION

Bacteriophages are highly specific viruses that infect bacteria. Following infection of a bacterium like E. Coli by a lytic phage, such as T4, a profound rearrangement of macromolecular syntheses occurs. In T4 infection, for example, the RNA Polymerase (RNAP) of the host bacterium binds to initiation sites of the phage genome known as Immediate-Early (IE) genes and transcribes them. Some IE gene products degrade the host (bacterial) DNA, which lacks the modified base Hydroxy Methyl Cytosine (HMC), while another product ADP-Ribose, binds to the alpha subunits of the bacterial RNAP and renders it incapable of recognizing bacterial cell promoters. This results in the cessation of transcription of host genes. These events occur in the first 3 to 5 minutes after infection.

In the next stage, the modified RNAP recognizes and binds to the so-called Delayed Early (DE) genes, thus eliminating further expression of the IE genes of the phage. The DE gene products are involved in replicating the phage genome using the degraded bacterial DNA bases. One of the products of the DE genes is a novel sigma factor that causes the host RNAP to recognize only the Late (L) genes which are the next to be transcribed. The Late genes are involved in synthesizing new capsid proteins, tails and tail fibers and assembly proteins, which are needed to assemble progeny phage particles. Finally, the phage lysozyme gene is activated resulting in the lysis of the bacterial host cell and release of the progeny phage. For a review of T4 phage biology and early events in T4 replication see, e.g., Miller et al. “Bacteriophage T4 genome”, Microbiol Mol Biol Rev. (2003) 67(1):86-156; Wilkens et al. “ADP-ribosylation and early transcription regulation by bacteriophage T4”, Adv Exp Med Biol. (1997) 419:71-82; Harvey Lodish et al., eds.; Molecular Cell Biology, Fourth Edition, (2000), W. H. Freeman, New York, N.Y. and Houndsmills, Basingstoke, England (particularly Chapter 6: Manipulating Cells and Viruses in Culture); Mathews (ed. 1983) Bacteriophage T4 Am. Soc. Microbiol.; Karan, et al. (eds. 1994) Molecular Biology of Bacteriophage T4 Am. Soc. Microbiol.: Snyder and Champness (2002) Molecular Genetics of Bacteria (2d ed.) Am. Soc. Microbiol.; Birge (2000) Bacterial and Bacteriophage Genetics Springer-Verlag; and Calendar (1988) The Bacteriophages (Viruses) Plenum.

In view of their highly specific lytic effect, bacteriophages acting on infectious pathogens have been investigated from the time of their discovery to the present day for their therapeutic potential. Bacteriophage preparations for treatment of bacterial infections (see, e.g., U.S. Pat. No. 6,121,036) and in inhibition of dental caries (U.S. Pat. No. 4,957,686) have been described. Although highly successful initially, phage therapy is controversial due to a historical lack of quality control, regulatory processes and inadequate understanding of the high specificity of phages for their bacterial hosts. Phage therapy was abandoned in the western world after the advent of antibiotics in the 1940s. However, in view of the emergence of antibiotic resistance in recent years, there is renewed interest in the development of alternative methods for treating infection, including phage therapy (Sulakvelidze et al. Antimicrob Agents Chemotherap, 45, 649, (2001)).

Although phage therapy has been attempted in various contexts for many years with variable success, several problems need to be addressed before phages can become acceptable therapeutic agents. Many of the problems encountered by the early investigators, such as contamination of phage preparations with host bacteria and bacterial debris, can be overcome by modem methodologies that have been developed in the past few decades. Basic properties of phages like rapid clearance by the spleen, liver and the reticulo-endothelial system, and the potential for development of antibodies in the human host during treatment, however, require novel solutions if phage therapy is to become generally applicable. One approach for addressing the first problem, namely, rapid clearance, was described by Merrill et al (Proc. Natl. Acad. Sci. USA 93, 3188 (1996); see also U.S. Pat. No. 5,688,501) which involved the selection of long-circulating variants of wild type phages by serial passage in animals.

The generation of neutralizing antibodies after the administration of phages to humans and animals is a major concern that hinders the development of phage therapy, especially for chronic infections. It has been reported that neutralizing antibodies appear a few weeks after the administration of phages to humans or animals (Slopek et al. Arch. Immunol. Ther. Exp., 35, 553(1987)). Administering higher doses of phage has been suggested as a possible solution (Carlton, R. M., Arch. Immunol. Ther. Exp., 47, 267(1999); however, this is not the most attractive of alternatives. For example, a high-dosing approach requires production of a far greater number of phage for each dose to be administered.

Many studies of potentially therapeutic phages to date have focused on the lytic endpoint that releases progeny phage which can invade other bacterial hosts and destroy them. This amplification provided by the lytic process in the bacterial host is an attractive feature of phage therapy, as it facilitates production of more phage and killing of infecting bacteria. However, phage amplification and release through lysis also exposes the subject being treated to a bolus of bacteriophage antigen. This poses the risk that the host will mount an immune response to the phage, which immune response may be undesirable, facilitate clearance of the phage, or both.

During the past decade, the key components essential for host lysis by bacteriophages have been investigated. It is now recognized that two proteins, an endolysin and a holin are needed for host lysis to occur. Endolysins are muralytic enzymes that accumulate in the cytosol and holins are small membrane proteins that regulate access of the endolysins to the cell wall through the cytoplasmic membrane (Wang et al., Ann. Rev. Microbiol.54, 799-825 (2000)). The lysis gene region of bacteriophage lambda was cloned into a multi-copy plasmid, pBH 20 under the transcriptional control of the lac operator and induction of this “lysis operon” led to lytic behavior parallel to that of bacteriophage infected cells (Garrett, J. et al. Mol. Gen. Genet. 182, 326(1981). The two lysis genes cph1 and cpl1 of the Streptococcal pneumoniae bacteriophage Cp-1, coding for holin and lysin respectively, have been cloned and expressed in E. coli (Martin et al. J. Bacteriol. 180, 210 (1998)). Expression of the Cph1 holin resulted in bacterial cell death but not lysis. Concomitant expression of both holin and lysin of phage Cp-1 in E. coli resulted in cell lysis. Furthermore, the cph1 gene was able to complement a lambda Sam mutation (carrying an amber mutation in the holin gene) in the nonsuppressing E. coli KB101 strain to release phage progeny. Regulated expression of lambda phage lysis genes S and R causes dramatic lysis of both Vibrio cholerae and Salmonella enterica serovar Typhimurium cells (Jain et al. Infect Immun, 68, 986 (2000).

There is a need in the field for methods and compositions to provide for therapeutic bacteriophage, e.g., having reduced immunogenicity in the host. The present invention addresses this and other needs.

Literature

Johnson-Boaz et al. Mol Microbiol. Aug. 13, 1994;(3):495-504

d'Herelle. Crit. Rev. Acad. Sci. Paris, 165, 373 (1917)

Blasi et al. “Two beginnings for a single purpose: the dual-start holins in the regulation of phage lysis” Mol Microbiol. Aug. 21, 1996;(4):675-82;

Sanders et al Appl Environ. Microbiol. 1997 63 4877-82;

Smith et al. “Purification and biochemical characterization of the lambda holin” J Bacteriol. 1998 May; 180(9):2531-40;

Martin et al. “Functional analysis of the two-gene lysis system of the Pneumococcal phage Cp-1 in homologous and heterolgous host cells” J. Bacteriol. 180:210-7 (1998);

Dressman et al. “Lysis and lysis inhibition in bacteriophage T4:rV mutations reside in the holin t gene” J Bacteriol. 1999 July;181(14):4391-6;

Blasi et al. “The C-terminal sequence of the lambda holin constitutes a cytoplasmic regulatory domain.” J Bacteriol. 1999 May;181(9):2922-9;

Vukov et al. “Functional analysis of heterologous holin proteins in a lambdaDeltaS genetic background.” FEMS Microbiol Lett. Mar. 15, 2000;184(2):179-86;

Wang et al. “Holins: the protein clocks of bacteriophage infections.” Annu Rev Microbiol. 2000;54:799-825;

Grundling et al. “Holins kill without warning” Proc. Natl. Acad. Sci. USA 98:9348-52 (2001);

Ramanculov et al. “Functional analysis of the phage T4 holin in a lambda context,” Mol. Genet. Genomics 265:345-53 (2001);

Ramanculov et al. “Genetic analysis of the T4 holin: timing and toplogy” Gene 265:25-36 (2001).

Tan et al. “Evidence for holin function of tcdE gene in the pathogenicity of Clostridium difficile.” J Med Microbiol. 2001 July;50(7):613-9;

Young, “Bacteriophage holins: deadly diversity.” J Mol Microbiol Biotechnol. Jan. 4, 2002;(1):21-36;

Hori et al Appl Microbio. Biotech 2002 59 211-21;

British patent no. GB829266A “A method of manufacturing bacteriophages in solid dry form”, published Mar. 3, 1960;

European publication no. EP0403292A2 “Ruminant feedstuff additives”, published Dec. 19, 1990;

U.S. Pat. No. 4,957,686, “Use of bacteriophage to inhibit dental caries”, issued Sep.18, 1990;

PCT Publication No. WO95/27043 “Antibacterial therapy with genotypically modified bacteriophage”, published Oct. 12, 1995;

PCT Publication NO. WO95/31562 “Process for inhibiting the growth of a culture of lactic acid bacteria, and optionally lysing the bacterial cells, and uses of the resulting lysed culture”, published Nov. 23, 1995;

U.S. Pat. No. 5,688,501, “Antibacterial therapy with bacteriophage genotypically modified to delay inactivation by the host defense system”, issued Nov. 18, 1997;

U.S. Pat. No. 6,121,036, “Compositions containing bacteriophages and methods of using bacteriophages to treat infections” issued Sep. 19, 2000;

U.S. Publication No. US2002/0058027 “C1 bacteriophage lytic system,” published May 16, 2002;

U.S. Pat. No. 6,264,945 “Parenteral use of bacterial phage associated lysing enzymes for the therapeutic treatment of bacterial infections,”issued Jul. 24, 2002;

PCT Publication No. WO01/82945 “The use of bacterial phage associated lysing enzymes for treating variously illnesses,” published Nov. 8, 2001.

Eaton et al. (1934). Bacteriophage Therapy. JAMA 103:1769-1776; 1847-1853; 1934-1939

Kutter, “Bacteriophage Therapy,” at www.evergreen.edu/phage/phagetherapy/phagetherapy.html, last updated Nov. 8, 2003)

Walker et al. (1980) “Mutation in coliphage T1 affecting host cell lysis,” J. Virol 35:519-530

### SUMMARY OF THE INVENTION

The present invention features composition and methods for treating a bacterial infection using therapeutic bacteriophage having a modified holin gene. The modified holin inactivates the bacterial host prior to production of bacteriophage, so that the bacteriophage infection is non-productive, e.g., few or no bacteriophage are produced as a result of infection of the bacterial host. Thus, holin-modified bacteriophage invade the bacterial host, and cause inactivation of the bacterial host prior to production of a detectable or significant number of phage. Holin-modified phage inhibit the spread of bacterial infection without production of a significant or detectable number of phage. By avoiding the release of phage progeny, the potential for generation of immune responses against the phage is reduced.

One feature of the invention is that it provides a general procedure to eliminate or minimize the development of an immune response against the phage when used for treating bacterial infection. Another feature of the invention is that it provides methods and compositions to treat bacterial infections, particularly infections by drug-resistant, pathogenic bacteria.

Still another feature of the invention is that it provides methods and compositions for use in antibacterial treatment of materials and surfaces in vitro.

One advantage of the invention is that the use of holin-modified bacteriophages provides for reduced clearance of the bacteriophage to allow for more effective therapy, while at the same time avoiding undesirable immune responses in the subject being treated. Infection of a pathogen with a holin-modified bacteriophage results in infection and inactivation (up to and including lysis) of the bacterial host. The bacterial pathogen infected with a holin-modified phage is at least rendered incapable of multiplying and spreading the bacterial infection, and preferably is killed, e.g., by inactivation, e.g., by rendering the bacterial host bacteriostatic, lysis, and the like. Use of holin-modified bacteriophage results in containing and ultimately eliminating the pathogen with reduced or no detectable release of phage into the environment or human host during treatment of the infection.

Another advantage of the invention is that the phage-infected bacteria are inactivated in a manner that will not provide for resumption of bacterial replication once therapy is terminated. This provides for control in dosing of the phage.

Still another advantage is that, since the holin-modified phage have reduced ability to replicate relative to a wild-type phage, and thus produce relatively few progeny in the course of use in the methods described herein, the likelihood of the generation and selection of reversion mutants is decreased.

These and other advantages and features of the invention will become apparent to those persons skilled in the art upon reading the details of the animal model and methods of its use as more fully described below.

Before the present invention is described, it is to be understood that this invention is not limited to particular methodology, protocols, bacteriophage, bacterial pathogens, animal species or genera, constructs, and reagents described, as such may, of course, vary. It is also to be understood that the terminology used herein is for the purpose of describing particular embodiments only, and is not necessarily intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a bacteriophage” includes a plurality of such bacteriophage and reference to “the host cell” includes reference to one or more host cells and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Although the potential of phage therapy for treating antibiotic resistance is generally acknowledged, the development of phage therapy has lagged due to the controversy surrounding the use of phages in the 1920s and 1930s, as well as concerns about the potential for immune responses against therapeutic phage. (Eaton et al. (1934). Bacteriophage Therapy. JAMA 103:1769-1776; 1847-1853; 1934-1939; see also Kutter, “Bacteriophage Therapy,” at www.evergreen.edu/phage/phagetherapy/phagetherapy.html, last updated Nov. 8, 2003).

The production of well-defined and well-characterized phage using modem technologies and current standards of quality control have addressed many of the issues that led to controversies about phage therapy in the past. However, the potential for eliciting immune responses is a fundamental property of bacteriophages and prevention of the immune response or reduction of this potential is important for effective application of phage therapy. The use of therapeutic bacteriophages in the treatment of bacterial infection is, in some regards, a race between the bacteriophage as it infects the bacteria of the subject's infection and the body's attempts to clear the bacteriophage from the body. Clearance can be accomplished by, for example, the “passive” clearance system of adsorption, metabolism, excretion by liver and kidneys and/or by the separate active “immune” response which produces antibodies which facilitate inactivation and clearance of subsequent exposures. The object of this invention is to provide a procedure to delay, minimize, or eliminate (avoid) the development of an immune response against the phage when it is used for treating bacterial infection.

The present invention accomplishes this objective by providing bacteriophage having a modified holin. Holin-modified bacteriophage can infect bacteria and inhibit bacterial growth, but cause inactivation of the bacterial host—e.g., through rendering the host cell bacteriostatic or through host cell lysis—prior to production of a significant number or any detectable phage particles in the host cell, e.g., prior to the assembly of complete phage particles in the phage replication cycle. The bacteriophages of the invention in essence act as antimicrobial agents that inhibit bacterial replication (including by killing the bacteria, e.g., through lysis), without significant production or release of bacteriophage particles. By avoiding bacteriophage production, or at least reducing the number of bacteriophage produced, the number of bacteriophage presented to the immune system of the subject undergoing therapy is exposed is significantly decreased (e.g., compared to therapy with a wild-type bacteriophage). Since the number of phage present in the host is reduced, the host immune response against the therapeutic bacteriophage is less robust, thus reducing the ultimate production of a humoral immune response, and the development of a high clearance rate of the therapeutic phage by antibody conjugation at a later date when antibodies might be produced.

Antibiotics exert their action either by killing the bacteria (bactericidal) or by inhibiting the growth of the bacteria (bacteriostatic). Although bactericidal agents are preferred, bacteriostatic agents have also been beneficial, since the normal defenses of the host can often shift the balance of destruction over replication, then destroy the slower growing bacterial population. Specific infection of a bacterial pathogen by genetically modified bacteriophage proposed in this invention provide for at least inactivation (e.g., bacteriostasis) of the pathogen, and in some embodiments killing (e.g., through lysis) of the pathogen. The inactivated or killed bacterial pathogens are eliminated by the normal clearance mechanisms of the host. In contrast to bacteriostatic antimicrobial agents in which withdrawal of therapy can lead to the resumption of the infection, phage-inactivated bacteria remain non-viable and cannot resume growth and progress of infection.

The holin-modified phage of the invention can be used to inactivate or kill a specific bacterial host and, therefore, can be developed as a therapeutic agent for the treatment of bacterial infection. The present invention is thus applicable to all bacteriophages which involve a holin as part of the lytic cycle or which can be modified to express a holin as described herein. Current literature indicates that all double-stranded DNA (dsDNA) phage have holins as well as endolysins (Wang et al, Annu Rev Microbiol. 2000;54:799-825).

Specific aspects of the invention will now be described in more detail.

Definitions

By “bacteriophage” and “phage”, which terms are used interchangeably herein, is meant any of a variety of viruses that have a specific affinity for and infect bacteria. These thus include, coliphages, which infect Escherichia coli (e.g., lambda phage and the T even phages, T2, T4 and T6). Phages generally are composed of a protein coat or capsid enclosing the genetic material, DNA or RNA, that is injected into the bacterium upon infection. In the case of virulent phages synthesis of host DNA, RNA and proteins ceases and the phage genome is used to direct the synthesis of phage nucleic acids and proteins using the host's transcriptional and translational apparatus. These phage components then self assemble to form new phage particles. The synthesis of a phage lysozyme leads to rupture of the bacterial cell wall releasing, typically, 100-200 phage progeny. The temperate phages, such as lambda, may also show this lytic cycle when they infect a cell, but more frequently they induce lysogeny, in which the phage integrates into the bacterial host DNA to persist as a prophage. In general, the bacteriophage of interest in the invention are lytic phages rather than temperate phages.

By “holin-modified phage” is meant a phage having a holin gene that is other than a holin gene endogenous to the typical (wild type) host phage genome (e.g., a holin of a different phage, a mutant holin, and the like), a holin-encoding sequence that is operably linked to a promoter that facilitates holin production early in the bacteriophage infection or replication cycle, e.g., at a stage earlier than that at which holin is normally produced, or both. Holin-modified bacteriophage infect a bacterial host cell and provide for expression of a modified holin gene so that the bacterial host cell is inactivated (e.g., rendered bacteriostatic) or lysed prior to assembly of any detectable or a significant number of phage particles. Further exemplary holin-modified phage are described below.

By “isolated” is meant that the material is at least 60%, by weight, free from the proteins and naturally-occurring organic molecules with which it is naturally associated. Preferably, the material is at least 75%, more preferably at least 90%, and most preferably at least 99%, by weight, the material of interest. “Isolated” thus encompasses preparations that are enriched for the desired material.

The terms “polynucleotide” and “nucleic acid”, used interchangeably herein, refer to polymeric forms of nucleotides, including ribonucleotides, deoxynucleotides, or mixed nucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide” and “protein”, used interchangeably herein, refer to a polymeric form of amino acids, which can include coded and non-coded amino acids, chemically or biochemically modified (e.g., post-translational modification such as glycosylation) or derivatized amino acids, polymeric polypeptides, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is normally associated in nature, (2) is linked to a polynucleotide other than that to which it is normally linked in nature, or (3) does not normally occur in nature.

“Recombinant phage”, “recombinant host cells”, “host cells”, “cells”, “cell lines”, “cell cultures”, and other such terms denoting microorganisms or higher eukaryotic cells cultured as unicellular entities refer to cells which can be, or have been, used as recipients for recombinant vector or other transfer DNA, and include the progeny of the original cell which has been transfected. It is understood that the progeny of a single parental phage or parental cell may not necessarily be completely identical in morphology or in genomic or total DNA complement as the original parent, due to natural, accidental, or deliberate mutation.

“Operably linked” refers to a juxtaposition wherein the components so described are in a relationship permitting them to function in their intended manner. A control sequence “operably linked”to a coding sequence is ligated in such a way that expression of the coding sequence is achieved under conditions compatible with the control sequences.

An “open reading frame” (ORF) is a region of a polynucleotide sequence which encodes a polypeptide; this region may represent a portion of a coding sequence or a total coding sequence.

A “coding sequence” is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, and recombinant polynucleotide sequences.

“Heterologous” means that the materials are derived from different sources (e.g., from different genes, different species, etc.).

“Transformation”, as used herein, refers to the insertion of an exogenous polynucleotide into a host cell, irrespective of the method used for the insertion, for example, direct uptake, transduction, f-mating or electroporation. The exogenous polynucleotide may be maintained as a non-integrated vector, for example, a plasmid, or alternatively, may be integrated into the host genome.

The terms “individual,” “subject,” “host,” and “patient,” are used interchangeably herein and refer to any subject having a bacterial infection amenable to treatment using the therapeutic bacteriophage of the invention, and for whom treatment or therapy is desired. Mammalian subjects and patients, particularly primate (including human) subjects or patients are of particular interest. Other subjects may include livestock and pets, e.g., cattle, pigs, sheep, chickens, dogs, cats, guinea pigs, rabbits, rats, mice, horses, and so on.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease (e.g., eliminating an infection, reducing the severity of an infection, reducing bacterial load, inhibiting growth of bacteria, etc.). “Treatment” as used herein covers any treatment of a disease in a subject, particularly a mammalian subject, more particularly a primate, including human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom.

By “infecting bacterium” is meant a bacterium that has established infection in the host, and which may be associated with a disease or undesirable symptom as a result. Generally, infecting bacteria are pathogenic bacteria, and occasionally the bacteria comprise a plurality of species which interact together to cause the pathology. In certain circumstances, inhibition of either or both of the interacting species may be sufficient or necessary to treat.

By “drug-resistant bacteria” or “antibiotic-resistant bacteria” is meant a bacterial strain that is resistant to growth inhibition or killing by an antibiotic. Multi-drug resistant bacteria are resistant to two or more antibiotics. Drug resistance can encompass, for example, ineffective killing of the infecting bacteria such that at least an infectious dose remains in the subject and the infection continues, resulting in continued symptoms of the associated infectious disease or later evidence of such symptoms. Drug resistance can also encompass inhibiting growth of the drug-resistant bacteria until such time therapy is discontinued, after which the bacteria begin to replicate and further the infectious disease.

By “inhibition of bacterial growth” in the context of infection of a bacterial cell with a holin-modified bacteriophage is meant that, following infection of the bacteria, the bacteriophage inhibits or interferes with the bacterial host cell's normal transcriptional and/or translational mechanisms such that the infected bacteria does not undergo substantial cell division (replication) and is caused to enter a state of bacteriostasis and/or is killed, e.g., by lysis.

Bacteriophage for Production of Holin-Modified Bacteriophage

A holin-modified phage of the invention can be generated from many wild-type bacteriophage, preferably from a lytic phage. Thus, a variety of holin-modified bacteriophages which are specific for a variety of bacteria, are thus useful in the treatment of a wide variety of bacterial infections. While it is contemplated that the present invention can be used to treat most any bacterial infection in an animal, the invention finds particular use in therapy (adjunctive or stand-alone) for infections caused by drug-resistant bacteria. Exemplary drug-resistant, clinically-important bacterial species and strains are listed below. The American Type Culture Collection (ATCC, Manassas, Md.) accession number for an exemplary wild-type bacteriophage infecting the corresponding clinically-relevant strains are provided following the strain it infects. Such phage are exemplary of those that can be engineered to be a holin-modified phage to provide the therapeutic bacteriophage according to the invention. An exemplary list is as follows, where clinically important bacteria include both human and non-human animal infections:

1. Clinically important members of the family Enterobacteriaceae, including, but not limited to:

a. Clinically important strains of Escherichia, with E. coli being of particular interest (ATCC phage#23723-B2);

b. Clinically important strains of Klebsiella, with K. pneumoniae (ATCC phage #23356-B1) being of particular interest;

c. Clinically important strains of Shigella, with S. dysenteriae being of particular interest (ATCC phage #11456a-B1);

d. Clinically important strains of Salmonella, including S. abortus-equi (ATCC phage #9842-B1), S. typhi (ATCC phage #19937-B1), S. typhimurium (ATCC phage #19585-B1, S. newport (ATCC phage #27869-B1), S.paratyphi-A (ATCC phage #12176-B1), S. paratyphi-B (ATCC phage #19940-B1), S. potsdam (ATCC phage #25957-B2), and S. pollurum (ATCC phage #19945-B1);

e. Clinically important strains of Serratia, most notably S. marcescens (ATCC phage #14764-B1)

f. Clinically important strains of Yersinia, most notably Y. pestis (ATCC phage #11953-B1)

g. Clinically important strains of Enterobacter, most notably E. cloacae (ATCC phage #23355-B1);

2. Clinically important Enterococci, most notably E. faecalis (ATCC phage #19948-B1) and E. faecium (ATCC phage #19953-B1)

3. Clinically important Haemophilus strains, most notably H. influenzae (exemplary phage can be obtained from the World Health Organization (WHO) or other labs that make them available publicly);

4. Clinically important Mycobacteria, most notably M. tuberculosis (ATCC phage #25618-B1), M avium-intracellulare, M. bovis, and M. leprae. (exemplary phage available commercially from WHO, via The National Institute of Public Healthy & Environmental Protection, Bilthoven, The Netherlands);

5. Neisseria gonorrhoeae and N. meningitidis (exemplary phage can be obtained publicly from WHO or other sources);

6. Clinically important Pseudomonads, with P. aeuruginosa being of particular interest (ATCC phage #14203-B1);

7. Clinically important Staphylococci, with S. aureus (ATCC phage #27690-B1) and S. epidermidis (exemplary phage available publicly through the WHO, via the Colindale Institute in London) being of particular interest;

8. Clinically important Streptococci, wit S. pneumoniae being of particular interest (exemplary phage can be obtained publicly from WHO or other sources); and

9. Vibrio cholera (phage #14100-B1)

In general, additional bacterial pathogens, far too numerous to mention here, can also be susceptible to therapy according to the present invention, e.g., bacterial pathogens of non-human animals (e.g., livestock, domestic pets, and the like). Thus the invention contemplates treatment of non-human animals as will, for which various bacterial pathogens can be targeted for therapy according to the invention. Of particular interest are bacteria in which drug-resistance has developed,

In short, bacterial infections caused by bacteria for which there is a corresponding holin containing phage either currently available or for which holin containing phage can be identified, can be treated according to the present invention by producing a holin-modified phage using the corresponding wild-type phage, and contacting the bacteria with the holin-modified phage.

Double-stranded DNA phage having a holin can also be used in the present invention. Isolation of phage from an environment of interest (e.g., hospitals, sewage and other sources) can be accomplished using =standard procedures (see, e.g., Seeley et al. J Appl Bacteriol. 1982 53(1):1-17). Typically, 9 ml of the sewage sample is mixed with 1 ml of 10X LB broth, then 0.1 ml of overnight LB broth shake culture growth of target bacterial strain is added and incubated overnight at 37° C. Chloroform (0.1 ml) is added and incubated at 37° C. for 15 minutes with shaking at 300 rpm. This is then centrifuged at 14,000 rpm for 20 minutes at 4° C. and the supernatant is stored in sterile Eppendorf tubes. These crude phage preparations are further purified and characterized as needed.

Production of Modified-Holin Phage

Generation of the modified-holin phage can be viewed as involving the following general steps: (1) selecting a bacterium that is to be the target of holin-mediated killing using a holin-modified phage; (2) selecting or identifying a phage which can infect the target bacterium; (3) generating a production host for propagation of a modified-holin phage, where the production host is resistant to modified holin-mediated inactivation; and (4) produce a phage having a modified holin by selection, with or without mutagenesis. In another embodiment, modified-holin phage can be generated by: (1) selecting a target bacterium; (2) selecting or identifying a phage which can infect the target bacterium; (3) generate a modified holin, e.g., using a plasmid release technique (see, e.g., Kloos et al. (1994) J. Bacteriol. 176:7352-61) so as to identify modified holins that mediate early host inactivation (e.g., early lysis); and (4) introduce the modified holin into the phage of interest by recombination. Production of holin-modified phage progeny can be accomplished using a recombinant production host bacterium, which is protected against early inactivation (e.g., by expression of, for example, an antiholin protein or antisense message against the modified holin) so as to allow for phage replication and production of modified-holin phage, and isolating modified-holin phage from the supernatant of the production host bacterium.

Holin-modified bacteriophage having desired characteristics compared to wild-type phage (e.g., release of fewer phage particles) can be verified by, for example, comparing the relative amount of phage progeny produced following infection of a bacterial host with a holin-modified phage compared to that produced following infection with the corresponding wild-type phage. The comparison of phage progeny produced can be accomplished by, for example, examining the relative amount of infectious phage particles produced, comparing amplification of phage in culture following infection, or comparing the amount of phage DNA present after a suitable period following infection of the bacterial host (e.g., by quantitative PCR or the like). In addition, holin-modified phage having the desired characteristics of reduced anti-phage immune response can be examined in a non-human animal model of bacterial infection.

Although the phage may be either lytic or lysogenic, it is generally preferred that therapeutic phage should not be lysogenic (temperate) phage. Production of holin-modified phage from lytic (non-lysogenic) phage, a production host can be provided, which production host allows efficient lytic propagation of the phage irrespective of the allelic character of the holin gene. This production host will be used both for the isolation and the propagation of the holin-modified phage.

In these embodiments, the objective is to produce a non-temperate phage which, in target host cells, undergoes abortive infection as a result of early holin gene function or early holin gene expression (i.e., as a result of a modified holin), resulting in a severe reduction in average yield of virions produced per infective cycle, and thus does not propagate effectively. In general, the holin-modified phage is propagated on a production host which inhibits the early function of the holin or endolysin genes and permits the infective cycles in the propagation (production) host to be productive rather than abortive, and thus allows production of the phage at useful levels. Thus, production of phage of the invention involves at least two components: the phage with the modified holin which causes abortive infection in target hosts, and the production host which inhibits the function of the modified holin and thus allows non-abortive propagation of the modified holin phage. Descriptions of both modified-holin phage and production hosts are provided below.

Modified Holin Phage

Modified holin for use in holin-modified phage can be generated in a variety of ways consistent with providing an infectious phage that causes inactivation of the host bacterium (e.g., by early lysis), preferably prior to production of infectious phage progeny. Holin-modified phage of the invention include those generated by introduction of a non-endogenous holin expression system, which provides for production of holin protein in the bacterial host at an early stage of phage infection, so that a sufficient amount of holin is present to inactivate or lyse the host cell prior to assembly of infectious phage.

For example, holin-modified phage facilitate inactivation (e.g., by rendering the host bacteriostatic, lysis, and the like) of the bacterial host so that the number of phage released (e.g., upon lysis of the bacterial host) is less than, preferably significantly less than, the number of phage that would be released at lysis following infection by the corresponding wild-type phage. For example, the holin-modified phage useful in the invention facilitate inactivation of the bacterial host, e.g., by lysis of the bacterial host, prior to accumulation of about 1, 2, 4, 6, 8, 10, 20, 30, 40, 50, or 75 phage per an infected bacterium (on average), or facilitates inactivation of the bacterial host, e.g., by lysis of the infected bacterial host, prior to production of more than about 2, 4, 6, 8, 10, 15, 20, 30, 40, 50, or 75 phage particles per bacterium infected by the bacteriophage (on average). Alternatively, the numbers of phage produced are less than about 2, 5, 8, 13, 17, 21, 26, 32, 37, 41, 47, 53, or 60% of the numbers of phage produced by wild type phage after a full cycle, or at a specific time point. Holin-modified phage useful in the invention facilitate holin-mediated killing of the bacterial host during or just after early gene expression, or within an early period during late gene expression, e.g., at about 1, 3, 5, 7, 10, 15, 20, 25, 30, 40, or 45 min after infection.

In general, a modified-holin gene construct can be prepared by isolating the sequences of the regions flanking a holin gene (about 100 bp on each side) of the phage to be modified. Generally, at least about 50 bp, 100 bp, 200 bp, 300 bp, 500 bp or more of homologous nucleic acid sequences are provided on each side, flanking the region of interest encoding the phage holin gene to be replaced (Singer (1982) Cell, 31: 25-33). For example, the DNAs corresponding to the upstream and downstream regions of each phage holin gene that is to be replaced by recombination can be isolated by nucleic acid amplification (e.g., PCR) and cloned into a plasmid having a selectable marker (e.g., ampicillin resistance) with a suitable restriction site between two regions for insertion of a DNA cassette into which the desired modified holin gene is inserted. This plasmid is introduced into appropriate bacterial host cells by transformation and selection for the selectable marker (exemplified here by ampicillin resistance). Alternatively, the construct of the plasmid may be genomically integrated in the bacterial host genomic DNA. This construct can then be used for generation of holin mutants or other modified-holin encoding constructs.

In one embodiment, an endogenous holin gene of a phage is modified so as to be operably linked to a promoter that facilitates early expression (e.g., expression of holin during early phage gene expression), e.g., by operably linking the holin gene to an early gene promoter, e.g., an immediate early gene promoter. In another embodiment, the phage is modified to provide an early promoter to a holin-encoding sequence that is not endogenous to the phage. The holin gene operably linked to an early expression promoter may be the endogenous holin, a wild-type non-endogenous holin, or a mutant holin, e.g., a holin modified to facilitate early host cell inactivation, e.g., lysis (discussed below).

In another embodiment, the promoter that facilitates early expression is an inducible promoter. Preferably, expression from the inducible promoter is induced by an agent that can be co-administered to a subject with the holin-modified phage, or which is induced by a bacterial host cell factor present in the target bacterium.

In another embodiment, the phage is modified to contain a mutant holin, which mutant holin has amino acid changes relative to a wild-type holin so that the mutant holin facilitates inactivation of the host cell (e.g., inhibition of host cell transcription, replication, and the like, which can include host cell lysis) prior to the accumulation of assembled phage particles in number associated with wild-type number. In general, mutant holin genes have at least one, or a combination of one or more, nucleic acid deletions, substitutions, additions, or insertions which result in an alteration in the corresponding amino acid sequence of the encoded lysin protein. For example, the mutant holin can be a mutant lambda holin, which mutant lambda holin have been described. See, e.g., the mutant holin S105, S_{A52G}, S_{C51S}, and S105_{CS15} mutant holin described in Grundling et al. Proc Natl Acad Sci 98:9348-52 (2001). Modification of the N and C terminal sequences of the lambda holin triggers early host cell lysis (see, e.g., Bläsi, et al. (1999). The C-terminal sequence of the lambda holin constitutes a cytoplasmic regulatory domain. J. Bacteriol. 181, 2922-2929; Steiner, et al. (1993). Charged amino-terminal amino acids affect the lethal capacity of lambda lysis proteins S107 and S105. Mol. Microbiol. 8, 525-533.

In lambda, a missense allele of S, Ala52Gly, causes lysis to occur prematurely at about 19-20 min after induction of a lysogen, compared to 45 min for the wild type. (Johnson-Boaz et al. Mol Microbiol. Aug. 13, 1994;(3):495-504). A lambda lysogen carrying this mutation begins to undergo lysis at 20 min after induction under standard inducing and growth conditions, as defined in the reference. Because the lysis of this lambda derivative occurs at about the time that the first progeny virion particle is assembled in the cell, this phage is essentially non-proliferative. On the average, under standard conditions, approximately 0.1 phage particles are produced per induced cell for this mutant, compared to approximately 50-100 for the isogenic parental. This phage can be produced by purifying the progeny virions from the lysate of the induced lysogens; from 10⁹ induced cells per ml, approximately 10⁸ virions per ml will be produced, or approximately 10¹¹ per liter. Thus holin-modified phages can be produced from induced lysogens, by direct concentration and purification.

Where necessary or desired, the mutant holin can be operably linked to an early promoter to provide for expression shortly after infection of the bacterial host cell.

A modified holin can also be generated using recombinant techniques such as site-directed mutagenesis (Smith Ann. Rev. Genet. 19, 423 (1985)), e.g., using nucleic acid amplification techniques such as PCR (Zhao et al. Methods Enzymol. 217, 218 (1993)) to introduce facile deletions, insertions and point mutations. Other methods for deletion mutagenesis involve, for example, the use of either BAL 31 nuclease, which progressively shortens a double-stranded DNA fragment from both the 5′ and 3′ ends, or exonuclease III, which digests the target DNA from the 3′ end (see, e.g., Henikoff Gene 28, 351 (1984)). The extent of digestion in both cases is controlled by incubation time or the temperature of the reaction or both. Point mutations can be introduced by treatment with mutagens, such as sodium bisulfite, which deaminates deoxycytidine to deoxyuridine resulting in the substitution of an A:T base pair for a G:C base pair in approximately 50% of the template molecules after one round of replication (Botstein et al. Science229, 1193 (1985)).

Other exemplary methods for introducing point mutations involve enzymatic incorporation of nucleotide analogs or misincorporation of normal nucleotides or alpha-thionucleotide by DNA polymerases (Shortle et al. Proc.Natl.Acad.Sci.USA79, 1588 (1982)). In oligonucleotide-directed mutagenesis, the target DNA is cloned into an M13 vector to produce single-stranded wild-type DNA template to which the oligo mutagen is annealed. This produces a noncomplementary (looped out) region on the oligo primer or on the template, resulting in an insertion or a deletion, respectively. Base pair mismatch between the template and the primer results in point mutagenesis. PCR-based mutagenesis methods (or other mutagenesis methods based on nucleic acid amplification techniques), are generally preferred as they are simple and more rapid than classical techniques described above (Higuchi et al. Nucleic Acids Res. 16, 7351 (1988); Vallette et al. Nucleic Acids Res. 17, 723 (1989)).

Holin-modified phage having a desired modified holin gene can be produced using marker rescue techniques. The technique of marker rescue has been used extensively to map mutations in phage, and to transfer artificially-generated mutations from phage genes cloned in a plasmid to the phage genome (Volker et al. Mol. Gen. Genet. 177, 447 (1980)). Exemplary of the use of this technique is the application to identify genes involved in T4 phage assembly and maturation. Specifically, restriction fragments containing the T4 phage assembly and maturation genes 20 to 22 were cloned in plasmids, mutagenized, and the mutations were then recombined back into the phage genome by infection of E. coli carrying the plasmid with a T4 20/21 am (amber) double mutant (Volker, supra, 1980). The phage progeny that had undergone recombination with the plasmid were selected by plating on a su⁻ host (lacking an amber suppressor) allowing the selection of recombinant phage. These am⁺ phages, were then screened non-selectively for the desired temperature-sensitive mutations in genes 20 and 21.

A similar strategy can be employed for construction of modified holin bacteriophage. A modified holin gene (either a modified or wild type (endogenous or non-endogenous) holin gene operably linked to an early expression promoter or inducible promoter), which can be generated using recombinant techniques described above, is cloned into a plasmid, preferably with a selectable marker, e.g., ampicillin-resistance. A bacterial host is selected which is susceptible to infection by a phage of interest, which phage is to be modified to be a holin-modified phage. A construct containing a modified holin (e.g., a mutant holin, a holin under control of a promoter to provide for early expression of holin, etc.) is introduced into the bacterial host. The recombinant bacterial host is then infected with the phage (e.g., a wild-type phage) of interest at a low multiplicity of infection. As the phage replicates, the phage recombine by a double crossover event with the modified holin gene in the bacterial host to yield holin-modified phage progeny. Propagation of holin-modified phage progeny for production purposes is described below.

Production Hosts for Holin-Modified Phages

Production hosts for generating holin-modified phage progeny is generally accomplished by providing for replication of holin-modified phage in a production host under conditions that avoid the activity of the modified holin of the phage in inactivation of the host early in the replication cycle. The conditions used for phage production in the production host are selected so that such conditions do not exist in the target bacteria. The “target bacteria” as used herein is the bacteria that the holin-modified phage is to inactivate in the methods described herein (e.g., an infecting bacteria in an infected host, a contaminating bacteria in a matrix to be sterilized, and the like).

There are at least two embodiments of the production host of particular interest: (1) a host which produces an anti-holin gene that inhibits or delays the function of the holin and (2) a host which produces anti-sense mRNA to the holin gene, such that the presence of the anti-sense mRNA inhibits or delays the expression of the holin gene.

In another example, a production host can suppress modified holin activity through expression of a factor which is not present in the target bacteria (e.g., an anti-holin, antisense, and the like). This holin suppression factor can be endogenous to the production host, or non-endogenous. In general the production host contains factors that suppress activity of the modified holin, or is cultured under conditions that retards the early inactivation activity of the modified holin.

In one example, the production host produces an antiholin protein that retards the action of the modified holin. Anti-holins have been described in the art (see, e.g., Ramanculov et al. “An ancient player unmasked: T4 rI encodes a t-specific antiholin,” Mol Microbiol. 2001 August;41(3):575-83; and Wang et al., Annu Rev Microbiol. 2000;54:799-825).

In another example, the production host expresses an antisense mRNA that inhibits synthesis of the modified holin of the phage that is to be produced in the cell. P Methods for accomplishing antisense-mediated inhibition of phage gene expression are known in the art (see, e.g., Walker et al. Appl Environ Microbiol. (2000) 66(1): 310-319).

Bacterial production hosts can be generated by using methods well known in the art. For example, where the production host is to express an anti-holin or an antisense mRNA, the anti-holin or antisense construct can be on a plasmid or genomically integrated. The anti-holin or antisense can be constitutively or inducibly expressed, as may be desired. Where the production host has an inducible construct encoding the holin suppression factor, the production host is grown under inducing conditions during infection with the holin-modified phage that is to be produced, so that the holin suppression factor (e.g., anti-holin or antisense mRNA) is expressed from the inducible promoter (e.g., in the presence of an inducing agent or under other conditions suitable for induction (e.g., temperature).

The production host is infected with holin-modified phage. Progeny of the holin-modified phage are then isolated form the production host after a time sufficient for phage particle formation. The production hosts can then be lysed to release the holin-modified phage. Phage progeny that accumulate in the production host may be released by, for example, mechanical (e.g., French press, freeze-thaw), enzymatic (e.g., lysozyme), or chemical (e.g., chloroform) means at the appropriate time. Alternatively, the bacterial production host can express a production holin, so as to provide for lysis and release of the phage at a time point late in the lytic cycle, e.g., at a time point associated with wild-type phage infection). Holin-modified phage progeny are then isolated from the production host culture.

Bacterial Infections Amenable to Bacteriophage Therapy

A variety of bacterial infections can be treated using a therapeutic bacteriophage according to the invention. The bacterial infection may be on the body surface, localized (e.g., contained within an organ, at a site of a surgical wound or other wound, within an abscess), or may be systemic (e.g., the subject is bacteremic, e.g., suffers from sepsis). Of particular interest is the treatment of bacterial infections that are amenable to therapy by topical application of the phage of the invention. Also of particular interest is the treatment of bacterial infections that are present in an abscess or are otherwise contained at a site to which the phage of the invention can be administered directly.

The subjects to be treated by the methods of the present invention include but are not limited to man or other primates, domestic pets, livestock, fish, and the animals in zoos, conservatories and aquatic parks (such as whales and dolphins).

The holin-modified bacteriophage of the present invention can be used as a stand-alone therapy or as an adjunctive therapy for the treatment of bacterial infections. Numerous antimicrobial agents (including antibiotics and chemotherapeutic agents and antibodies) are known in the art which would be useful in combination with holin-modified bacteriophage for treating bacterial infections. Examples of suitable antimicrobial agents and the bacterial infections which can be treated with the specified antimicrobial agents are listed below. However, the present invention is not limited to the antimicrobial agents listed below as one skilled in the art could easily determine other antimicrobial agents useful in combination with holin-modified bacteriophage.

| Pathogen | Antimicrobial or antimicrobial group |
|---|---|
| E. coli | trimethoprim-sulfamethoxazole (abbrev. TMO-SMO), or |
| uncomplicated urinary tract | ampicillin; 1st generation cephalosporins, |
| infection | ciprofloxacin |
| E. coli systemic infection | ampicillin, or a 3rd generation cephalosporin; |
| | aminoglycosides, aztreonam, or a penicillin + a |
| | pencillinase inhibitor |
| Klebsiella pneumoniae | 1st generation cephalosporins; 3rd generation |
| | cephalosporins, cefotaxime, moxalactam, amikacin, |
| | chloramphenicol |
| Shigella (various) | ciprofloxacin; TMO-SMO, ampicillin, |
| | chloramphenicol |
| Salmonella typhi | chloramphenicol; ampicillin or TMO-SMO |
| Salmonella non-typhi | ampicillin; chloramphenicol, TMO-SMO, |
| species | ciprofloxacin |
| Yersinia pestis | streptomycin; tetracycline, chloramphenicol |
| Enterobacter cloacae | 3rd generation cephalosporins, gentamicin, or |
| | tobramycin; carbenicillin, amikacin, aztreonam, |
| | imipenem |
| Haemophilus influenzae - | chloramphenicol or 3rd generation cephalosporins; |
| meningitis | ampicillin |
| Haemophilus influenzae - | ampicillin; TMO-SMO, cefaclor, cefuroxime, |
| other H. influenza infections | ciprofloxacin |
| Mycobacterium tuberculosis | isoniazid (INH) + rifampin or rifabutin, the above |
| and M. avium-intracellulare | given along with pyrazinamide +/or ethambutol |
| Neisseria meningitides | penicillin G; chloramphenicol, or a sulfonamide |
| Neisseria gonorrhoeae: | penicillin G; spectinomycin, ceftriaxone |
| penicillin-sensitive | |
| Neisseria gonorrhoeae: | Ceftriaxone; spectinomycin, cefuroxime or cefoxitin, |
| penicillin-resistant | ciprofloxacin |
| Pseudomonas aeruginosa | tobramycin or gentamycin (+/− carbenicillin, |
| | aminoglycosides; amikacin, ceftazidime, aztreonam, |
| | imipenem |
| Staphylococcus aureus: | penicillin G; 1st generation cephalosporins, |
| non-penicillinase-producing | vancomycin, imipenem, erythromycin |
| Staphylococcus aureus: | a penicillinase-resisting penicillin; 1st generation |
| penicillinase-producing | cephalosporins, vancomycin, imipenem, erythromycin |
| Streptococcus pneumoniae | penicillin G; 1st generation cephalosporins, |
| | erythromycin, chloramphenicol |
| Vibrio cholera | tetracycline; TMO-SMO |

Bacteriophage(s) suitable for use in treatment of a subject can be selected based upon the suspected bacterial pathogen infecting the subject. Methods for diagnosis of bacterial infections and determination of their sensitivities are well known in the art. Where such diagnosis involves culturing a biological sample from the subject, the clinician can at the same time test the susceptibility of the infecting pathogen to growth inhibition by one or more therapeutic phages that are candidates for subsequent therapy.

Efficacy of the bacteriophage therapy according to the invention can be monitored according to methods well known in the art. In general, successful treatment is that which results in inhibition of bacterial growth so as to allow the immune system of the infected host to facilitate clearance of the infecting bacteria., thereby reducing the bacterial load in the host. The holin-modified phage of the invention provide for reduced phage particles produced by holin-modified phage following infection of the host bacteria. These reduced phage progeny can translate to lower phage antigen doses presented to the host immune system, including protein carbohydrate, lipid and nucleic acid antigen, and thus less robust immune response directed against the phage. In addition, fewer phage progeny also statistically lowers the risk of gene transfer between bacterial hosts and thus between infected subjects, e.g., transfer of antibiotic resistance or other (e.g., of pathogenic) genes or prophage.

In addition to their therapeutic uses in vivo, the bacteriophage of the invention can also be used to generate an incapacitated whole cell bacterial vaccine, as described in commonly owned U.S. provisional application serial No. 10/259,164, filed Sep. 27, 2002, and in PCT publication no. WO 03/026690. By “incapacitated” is meant that the bacterial cell is in a state of irreversible bacteriostasis. While the bacterium retains its structure—and thus retains the immunogenicity, antigenicity, and receptor-ligand interactions associated with a wild-type bacterium—it is not capable of replicating due to the presence of an infecting phage with in the bacterial cell. Such vaccines are useful in eliciting a prophylactic or therapeutic immune response against the bacterial pathogen from which the vaccine is made. The holin-modified phage of the invention can be used to provide for early production of holin in the host bacterial cell, so that the bacterial cell is rendered incapacitated, but it not yet lysed.

Formulations, Routes of Administration and Dosages

The bacteriophage of the invention can be formulated in a manner suitable which provides for delivery of the bacteriophage to the site of infection, and which maintains the ability of the phage to infect and inhibit replication of the bacterial host cell.

Formulations and Pharmaceutical Compositions

The invention further contemplates pharmaceutical compositions comprising at least one bacteriophage of the invention provided in a pharmaceutically acceptable excipient. The formulations and pharmaceutical compositions of the invention thus contemplate formulations comprising an isolated bacteriophage specific for a bacterial host; a mixture of two, three, five, ten, or twenty or more bacteriophage that infect the same bacterial host; and a mixture of two, three, five, ten, or twenty or more bacteriophage that infect different bacterial hosts or different strains of the same bacterial host (e.g., a mixture of bacteriophage that collectively infect and inhibit the growth of multiple strains of Staphylococcus aureus). In this manner, the compositions of the invention can be tailored to the needs of the subject to be treated.

Various pharmaceutically acceptable excipients are well known in the art. As used herein, “pharmaceutically acceptable excipient” includes a material which, when combined with an active ingredient of a composition, allows the ingredient to retain biological activity and without causing disruptive reactions, e.g., with the subject's immune system.

The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). Further formulation guidance, e.g., for preparation and administration of formulations, is provided in, for example, Frkjr, et al. Pharmaceutical Formulation Development of Peptides and Proteins, Taylor & Francis (December 1999); Gibson Pharmaceutical Preformulation and Formulation: A Practical Guide from Candidate Drug Selection to Commercial Dosage Form CRC Press; (August 1, 2001); Mollet, et al. Formulation Technology: Emulsions, Suspensions, Solid Forms, Wiley & Sons; (January 23, 2001); Carpenter and Manning Rational Design of Stable Protein Formulations: Theory and Practice (Pharmaceutical Biotechnology, V. 13) Plenum Pub Corp; 1st edition (April 2002); Fletcher, et al. Practice and Principles of Pharmaceutical Medicine, Wiley & Sons; (Aug. 21, 2002); Zeng, et al. Particulate Interactions in Dry Powder Formulations for Inhalation, Taylor & Francis; 1st edition (Jan. 15, 2001); Finlay The Mechanics of Inhaled Pharmaceutical Aerosols: An Introduction Academic Press; (Jul. 4, 2001).

Exemplary pharmaceutically carriers include sterile aqueous of non-aqueous solutions, suspensions, and emulsions. Examples include, but are not limited to, any of the standard pharmaceutical excipients such as a phosphate buffered saline solution, water, emulsions such as oil/water emulsion, and various types of wetting agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

A composition comprising a bacteriophage of the invention may also be lyophilized using means well known in the art, for subsequent reconstitution and use according to the invention.

Also of interest are formulations for liposomal delivery, and formulations comprising microencapsulated bacteriophage. Compositions comprising such excipients are formulated by well known conventional methods (see, for example, Remington's Pharmaceutical Sciences, Chapter 43, 14th Ed., Mack Publishing Col, Easton Pa. 18042, USA). Also of interest are sustained release formulations of phage, which are adapted for implantation at a site (e.g., at or near a site of infection). The sustained release formulations can be adapted to provide for controlled release of phage over a desired period of therapy. Formulations of interest include gels, suspensions, and the like.

In general, the pharmaceutical compositions can be prepared in various forms, such as granules, tablets, pills, suppositories, capsules (e.g. adapted for oral delivery), microbeads, microspheres, liposomes, suspensions, salves, lotions and the like. Pharmaceutical grade organic or inorganic carriers and/or diluents suitable for oral and topical use can be used to make up compositions comprising the therapeutically-active compounds. Diluents known to the art include aqueous media, vegetable and animal oils and fats. Stabilizing agents, wetting and emulsifying agents, salts for varying the osmotic pressure or buffers for securing an adequate pH value.

The pharmaceutical composition can comprise other components in addition to the bacteriophage. In addition, the pharmaceutical compositions may comprise more than one bacteriophage, for example, two or more, three or more, five or more, or ten or more different bacteriophage, where the different bacteriophage may be specific for the same or different bacteria. For example, the pharmaceutical composition can contain multiple (e.g., at least two or more) defined holin-modified bacteriophage, wherein at least two of the phage in the composition have different bacterial host specificity. In this manner, the holin-modified bacteriophage composition can be adapted for treating a mixed infection of different bacteria, e.g., by selecting different groups of bacteriophage of differing specificity so as to contain at least one bacteriophage for each bacteria (e.g., strain, species, etc.) suspected of being present in the infection (e.g., in the infected site). As noted above, the bacteriophage can be administered in conjunction with other agents. For example, the phage can be administered in conjunction with a conventional antimicrobial agent (see table above). In another example, where the phage are administered by a nasal route to treat a sinus infection, the phage can be administered in conjunction with a decongestant or other agent suitable for use in treating a sinus infection or its symptoms. In some embodiments, it may be desirable to administer the bacteriophage and antibiotic within the same formulation, or as separate formulations. In some embodiments it may also be desirable to administer an antibacterial antibody. Where such additional antibacterial agents may be desired for administration, such can be administered before, with (e.g., at the time of), or following phage administration.

Routes of Administration and Dosages

The route of administration and dosage will vary with the infecting bacteria, the site and extent of infection (e.g., local or systemic), and the subject being treated. The routes of administration include but are not limited to: topical (e.g., to skin, eyes, and other exposed surface); oral, aerosol or other device for delivery to the lungs, nasal spray, nasal drops, intravenous (IV), intramuscular, intraperitoneal, vaginal, rectal, lumbar puncture, intrathecal, and direct application to the brain and/or meninges. The phage of the invention can also be administered by infusion (e.g., intravenous, subcutaneous, etc.), which may be desirable in the case of a localized infection. Excipients which can be used as a vehicle for the delivery of the phage will be apparent to those skilled in the art. For example, the free phage can be in lyophilized form and be dissolved just prior to administration by IV injection. The dosage of administration is contemplated to be in the range of about 1 million to about 10 trillion/per kg/per day, and preferably about 1 trillion/per kg/per day, and may be from about 10⁶ pfu/kg/day to about 10¹³ pfu/kg/day.

The phage are administered until successful elimination of symptoms from the pathogenic bacteria, which can be accompanied by inactivation and clearance of the bacteria, is achieved. Thus the invention contemplates single dosage forms, as well as multiple dosage forms of the compositions of the invention, as well as methods fro accomplishing delivery of such single and multi-dosages forms.

With respect to the aerosol administration to the lungs, the holin-modified phage is incorporated into an aerosol formulation specifically designed for administration to the lungs by inhalation. Many such aerosols are known in the art, and the present invention is not limited to any particular formulation.

Non-Therapeutic Uses of Holin-Modified Phase

In addition to the therapies described above, the holin-modified phage of the invention can also be used in applications in which it is used as an antibacterial agent, e.g., to facilitate sterilization of a physical environment. For example, the phage of the invention can be applied to a matrix, to provide for inactivation of bacteria in the matrix. “Matrix” as used herein refers to any physical environment or material in which inactivation of bacterium (e.g., sterilization) is desired. A “non-aqueous matrix” is of particular interest, which matrix is a solid or semi-solid matrix (e.g., a porous or non-porous substrate). Exemplary matrices include surfaces (e.g., floors, table tops, counters, shower facilities, medical facilities (e.g., operating rooms), surgical instruments, and the like); liquids (e.g., water and other liquids (e.g., for human or other animal consumption, e.g., milk, juice, and the like); solids or semi-solids, or mixtures thereof (e.g., manure, food products for human or other animal consumption); and the like. Also of interest are bandage materials (e.g., wound dressing materials, slings, and the like). Additional matrices of interest for treatment according to the invention include materials containing natural fibers, synthetic fibers, or both, including, for example, bedding (e.g., linens, sheets, blankets, towels, and the like), clothing, toys (e.g., plush toys), and the like.

Application of bacteriophage in such non-therapeutic uses can be accomplished by, for example, spraying or flooding a surface to be treated with a solution of the bacteriophage; mixing bacteriophage with the solid or semi-solid; and the like so as to provide for contact of the bacteria with an amount of bacteriophage effective to facilitate inactivation of the contaminating bacteria (e.g., inactivation of at least 50%, 60%, 75%, 85%, 90%, or 95% or more of the contaminating bacteria).

### EXAMPLES

### Example 1

### Anti-Holin Production Bacterial Host Strains for Use Production of Holin-Modified Phage

A production host is produced by first identifying the lysis genes of the phage that is to be the basis of the holin-modified phage and, by using genetics, genomics and/or physiological experimentation, the holin and antiholin genes are identified. The antiholin gene is cloned in an expression vector appropriate for the production host bacterial species; usually, this vector is a “shuttle plasmid” which can be propagated and engineered in E. Coli but which has an origin of replication, selectable genetic marker and promoter appropriate for the species of the production host.

By cloning the antiholin gene under a promoter that will function in the target species, a construct plasmid is produced which, when expressed in the production host, inhibits, at a functional level, the holin gene of the phage. The expression signals (e.g., promoter strength and ribosome binding site) and the plasmid copy number are the parameters which, for expression vector systems in all known bacterial hosts, can be directly manipulated using standard methods of site-directed and random mutagenesis, or by switching between plasmid origins of different copy number.

The plasmid is then engineered with the goal of finding a plasmid that prevents or severely delays lysis by the parental phage. The primary construct plasmid, and variants thereof modified to change the expression level of the antiholin, are tested in the target species by conducting single-step growth experiments, which determine lysis time and burst size (virion yield per infected cell). If substantial delay or complete inhibition is not achieved by optimizing the expression level of the antiholin, taking into account the consensus promoter and ribosome-binding site sequences and any other factors known to influence gene expression in the target species, then mutagenesis of the antiholin gene will be undertaken with the goal of increasing the inhibitory character of the antiholin protein. Based on the topology of known antiholins (i.e., the S107 antiholin of lambda, the gpRI antiholin of phage T4, the S²¹₇₁ antiholin of phage 21, or the LydB antiholin of phage P1), site-directed mutagenesis may be used to alter the sequence of the antiholin gene in ways that would be predicted to increase, based on knowledge of these other antiholins, would increase inhibitory capacity. Each construct is screened for increased delay or inhibition of holin function, as described above for the parental and expression-level variants.

Alternatively, mutant antiholin genes with increased inhibitory character can be selected. This is accomplished by altering the plasmid with appropriate random or localized mutagenesis techniques, transforming a pool of altered plasmids back into the target host strain, and subjecting the transformants to a “plasmid release” selection (see, e.g., Kloos et al. (1994) J. Bacteriol. 176:7352-61). In this case, the pooled transformants are infected with the parental phage and allowed to undergo a round of infection by the phage.

All the infected cells with plasmids that do NOT cause a delay in lysis undergo lysis; but plasmids with alterations that cause a delay of lysis or a blockage of lysis remain intact until the desired time of sampling and can be isolated by rapid filtering onto a sterilizing filter. The retained cells can then be resuspended in medium and lysed by mechanical or enzymatic means and the plasmid DNA isolated by standard plasmid isolation techniques (e.g., Qiagen quick-preparations). Candidate plasmids are transformed into the target species and are screened as above for site-directed mutants. If a high background of false-positives is obtained, candidates are pooled and selected to a second round of plasmid-release. In this way mutant antiholins with improved capacity for inhibiting holin function are obtained.

In some cases, antiholin genes can be constructed from holin genes. For example, deletion of the first transmembrane domain of the lambda S105 holin creates a defective holin which blocks function of the parental S105 holin and also delays function of the S105A52G abortive infection holin described above.

### Example 2

### Anti-Sense Production Bacterial Hosts for Producing Holin-Modified Phage

In this embodiment, an expression plasmid is engineered to produce anti-sense mRNA that anneals to the mRNA of the holin gene and block or severely reduce its expression. The simplest version of this embodiment is the cloning of the holin gene in inverted orientation to the promoter, such that a mRNA species is transcribed which is complementary to the entire coding sequence, nearby flanking sequences and translational initiation site of the holin gene.

This construct is transformed into the target species and then tested, as described above, for its ability to delay or abolish holin function, as indicated by the single-step growth kinetics, for the parental phage. Modifications to optimize or increase the level of the anti-sense mRNA can include improving the consensus match of the vector promoter, deleting portions of the complementary sequence, or inserting secondary structure sequences considered to be RNA-stabilizing elements to flank the anti-sense sequence, or a combination thereof.

### Example 3

### Production of a Holin-Modified Phage

Once a production host is constructed carrying a plasmid that inhibits holin gene expression or function, as described above, the holin-modified phage may be isolated or constructed. Holin gene mutations can be obtained which decrease the lysis time in the parental target host bacterium, to the extent that phage propagation was blocked or severely impaired, but would have a much less severe effect on the production host, carrying the inhibitory plasmid.

The simplest method relies upon the plating characteristics of the phage on the production host. If the parental phage is sufficiently inhibited, it may not make plaques on a lawn of the production host. Then mutant phage can be isolated that overcome the block in holin function simply by isolating rare revertant plaque-formers. Each candidate plaque-former is then tested for its ability to plate on the target species and for its lysis timing as described above. It is expected that some of the holin gene mutations which allow the phage to overcome the block in holin function or expression in the production host will be mutations that cause abortive infection in the target host lacking the inhibitory plasmid. If the spontaneous mutation frequency is not sufficiently high, then standard mutagenesis techniques can be applied to the phage (e.g., hydroxylamine mutagenesis).

The direct selection by plaque-formation may be impractical if the parental phage forms plaques on the production strain despite the inhibition of holin function or expression. This may come about because the inhibition of holin function in infections of the parental phage cause hyper-accumulation of phage particles during the extended infective cycle, such that although lysis is severely delayed, and thus fewer cycles are completed during the plating assay period, it is compensated by a much higher burst size per infected cell.

In this case, holin mutants can be isolated by mutagenzing the phage, infecting the mutagenized phage stock into the production host at a multiplicity of less than 1 (so most cells are infected by one phage, or none at all), and then taking samples of the culture medium at various times after infection (by rapid filtration). Any “early-lyser” phage with a holin mutation that accelerates lysis in the production host will be present in the medium at a much earlier time than the bulk of the parental phages. If necessary, phages isolated as “early-lysers” can be pooled and subjected to a second round of “early-lyser” selection on the production host.

Each “early-lyser” is tested for plaque-forming ability on the target host; candidates which fail to form plaques at unit efficiency on the target lawn but makes plaques on the production host becomes a candidate “holin-modified” phage. Each such candidate is tested in the target host by single-step growth and burst size assays, using the production host as a titering lawn. It is expected that many, and perhaps all mutants that significantly shorten the length of the vegetative cycle in the production host, which provides a block to holin function or expression, will be holin mutants with early lysis times, and in some cases, as in the case of Sa52g of lambda, the lysis time will be so early that the infection will be abortive (on the target species.)

### Example 4

### Administration of Holin-Modified Phage to Treat Bacterial Infection

Holin-modified phage can be screened for efficacy in an appropriate non-human animal model of bacterial infection. Successful treatment of experimental Escherichia coli infections in a mouse model has been described (see, e.g., Smith et al. J Gen Microbiol., 1982, 128:307-318; Bull et al., BMC Microbiology, 2002, 2:35).

The purified holin-modified phage are administered to a non-human animal model. For example, where the holin-modified phage are infectious for E. coli, purified holin-modified phage are administered to the animal model of Smith et al. (supra).

That which is claimed is:

1. A method for inhibiting growth of a bacterium, the method comprising:
contacting a bacterium with a holin-modified bacteriophage in an amount effective to infect the bacterium;
wherein said contacting results in infection of the bacterium by the holin-modified bacteriophage and inactivation of the bacterium.

2. The method of claim 1, wherein the bacterium in present in or on a non-aqueous matrix.

3. The method of claim 2, wherein the matrix is a solid substrate chosen from cloth, bandage material, a porous substrate, or a non-porous substrate.

4. A method for inhibiting growth of bacteria in an infected subject, the method comprising:
administering to the infected subject a holin-modified bacteriophage, wherein the bacteriophage is administered in an amount effective to infect a bacterium present in a subject and inhibit replication of the bacterium;
wherein said administering is effective to inhibit growth of the bacteria in the subject.

5. The method of claim 4, wherein:
a) the holin-modified bacteriophage comprises a mutant holin gene;
b) the bacteria in the host are drug resistant bacteria;
c) the bacterial infection is present on a body surface; or
d) the bacterial infection is at a local site.

6. The method of claim 4, wherein:
a) said administering is to the local site of infection;
b) at least two or more different holin-modified bacteriophage are administered to the subject, including wherein at least two of the holin-modified bacteriophage have specificity for different bacterial host cells; or
c) the subject has a mixed bacterial infection.

7. The method of claim 4, wherein the infecting bacteria is of a genus selected from the group consisting of Mycobacteria, Staphylococci, Vibrio, Enterobacter, Enterococcus, Escherichia, Haemophilus, Neisseria, Pseudomonas, Shigella, Serratia, Salmonella, Streptococcus, Klebsiella and Yersinia.

8. The method of claim 4, wherein the method further comprises administering an antimicrobial agent to the subject.

9. The method of claim 8, wherein the antimicrobial agent is administered prior to administration of the bacteriophage.

10. A pharmaceutical composition comprising a holin-modified bacteriophage and a pharmaceutically acceptable carrier suitable for administration to a human subject.

11. The composition of claim 10, wherein the bacteriophage is in lyophilized form.

12. The composition of claim 10, wherein said composition comprises a mixture of two or more holin-modified bacteriophage.

13. The composition of claim 12, wherein said composition comprises a mixture of two or more different holin-modified bacteriophage that effect inhibition of at least two different bacterial hosts.

14. A method of making a holin-modified phage, the method comprising:
contacting a bacterial production host with a holin-modified bacteriophage, wherein the bacterial production host suppresses early lysis activity of modified holin of the bacteriophage;
wherein infection of the bacterial production host with the holin-modified bacteriophage provides for production of holin-modified bacteriophage progeny.

15. The method of claim 14, wherein the production host suppresses modified holin activity by expression of an anti-holin.

16. The method of claim 14, wherein the production host suppresses modified holin activity by expression of an antisense mRNA which inhibits production of the modified holin.
